(19) United States
(12) Patent Application Publication
(10) Pub. No.: US 2011/0087250 A1
(43) Pub. Date: Apr. 14, 2011

(54) Gastric ring with switching pockets

(75) Inventors: Jean-Paul Ricol, Saint Germain sur l'Arbresie (FR); Pascal Paganon, Serezin du Rhone (FR)

(73) Assignee: Compagnie Europeenne d'Etude et de Recherche de Recherhe de Dispositifs pour I'lmplantation par La, Vienne (FR)

(21) Appl. No.: 12/937,391

(22) PCT Filed: Apr. 10, 2009
(86) PCT No.: PCT/FR2009/050676

§ 371 Date: Dec. 23, 2010

(30) Foreign Application Priority Data
Apr. 14, 2008 (FR) 08 02053

(51) Int. Cl. A61B 17/00

(52) U.S. Cl. 606/151

(57) ABSTRACT

The invention relates to an implantable surgical ring (1) to be placed around a biological organ while winding around a mean extension axis (X-X′), said ring comprising at least one first bearing part (5) designed to bear against the biological organ and capable of extending inside the ring (1) along a substantially radial first direction (D1), said ring being characterized in that said first bearing part (5) is defined by a first membrane (10) provided with an asymmetrical bendable structure designed, when the first bearing part (5) expands along the first direction (D1), to cause an accompanying shift of at least part of the first membrane (10) from one side to the other of a plane (P1) medial to the first bearing part (5) and normal to the extension axis (X-X′). The invention can be used in gastroplastic rings.

### TECHNICAL FIELD

The present invention pertains to the general technical field of surgical implants designed to be placed in a patient's body around a biological organ constituting a pocket or a duct, and especially gastric rings designed to constrict the stomach in a context of a treatment of obesity.

The present invention pertains more particularly to an implantable surgical ring designed to be placed around a biological organ constituting a pocket or a duct in order to modify the section of passage of said biological organ, said ring comprising at least one first contact piece designed to push against the biological organ and being capable of stretching into the ring in a first appreciably radial direction.

### PRIOR ART

There are known ways of practicing surgical operations on patients suffering from severe obesity who, because of their excess weight, are exposed not only to physical discomfort but also to psychological burdens as well as ancillary illnesses such as diabetes, cardiovascular illnesses or even severe arthritis.

In particular, a prior art technique consists in making a gastric constriction to reduce the size of the stomach and therefore the consumption of foodstuffs.

To this end, frequent recourse is had to gastroplasty rings implanted around the patient's stomach in order to reduce its volume as well as the diameter of its passage (namely the stoma).

Such gastroplasty rings generally comprise a flexible band made of elastomer material and designed to be closed at both ends by appropriate closing means suited to gripping the stomach.

Furthermore, the known rings usually comprise a ring-shaped compression chamber situated on the internal face of the flexible band, with a volume that can be adjusted by the addition or removal of a filling fluid.

In general, the prior-art implantable rings are satisfactory but sometimes have certain drawbacks.

In particular, the inflation of the compression chamber sometimes prompts an uneven deformation of its surface and especially the formation of folds which could damage the stomach wall by pinching or abrasion.

Furthermore, the inflation of the compression chamber may give rise to a certain fatigue in the material constituting the wall of said chamber, especially by distension of this chamber, thus reducing the lifetime of the ring.

Finally, it sometimes happens that the prior-art rings move accidentally under the effect of the natural movements of the stomach in such a way that the ring loses all or part of its therapeutical efficicacy and a new surgical operation has to be performed.

### SUMMARY OF THE INVENTION

The objects assigned to the present invention therefore seek to overcome the above-mentioned drawbacks and propose a novel implantable surgical ring, especially a gastric ring, possessing high capacities of adapting to varied conditions of use, while at the same time being particularly careful with the biological organ under treatment.

Another object assigned to the invention aims to propose a novel implantable surgical ring having a structure that is both simple and robust, giving it great longevity.

Another object assigned to the invention aims to propose a novel implantable surgical ring that presents a reproducible and predictable dynamic behavior and is particularly stable once implanted.

The objects assigned to the invention are achieved by means of an implantable surgical ring designed to be placed around a biological organ constituting a pocket or a duct, in getting wound around a mean axis of extension (X-X′) in order to modify the section of passage of said biological organ, said ring comprising at least one first contact piece designed to push against said biological organ and capable of extending into the interior of the ring along a first appreciably radial direction, said ring being characterized in that said first contact piece is demarcated by a first membrane provided with a deformable structure that is asymmetrical relative to said first direction and is designed, during the expansion of the first contact piece along the first appreciably radial direction, to bring about the joint migration of at least one part of the first membrane from one side to the other in a median plane of said first contact piece perpendicular to the axis of extension (X-X′).

### BRIEF DESCRIPTION OF THE DRAWINGS

Other features, characteristics and advantages of the invention shall appear in greater detail from the following description, as well as from the appended drawings, given purely by way of a non-restrictive illustration, of which:

FIG. 1 is an illustration, in a perspective view, of one alternative embodiment of the ring according to the invention in a closed configuration.

FIG. 2 provides an illustration, in a cut-away perspective view, of a portion of the ring illustrated in FIG. 1.

FIG. 3 is an illustration, in a cross-sectional schematic view, of a section of the ring shown in FIGS. 1 and 2 in a contracted configuration on the one hand and a deployed configuration on the other hand.

### BEST WAY TO ACHIEVE THE INVENTION

The first invention pertains to an implantable surgical ring 1 to be placed around a biological organ (not shown) constituting a pocket or a duct in order to modify the section of passage of said biological organ.

To this end, the ring 1 preferably has a flexible band 2 provided with junction means 3, 4 designed to close said flexible band on itself so that it forms a closed loop around the biological organ.

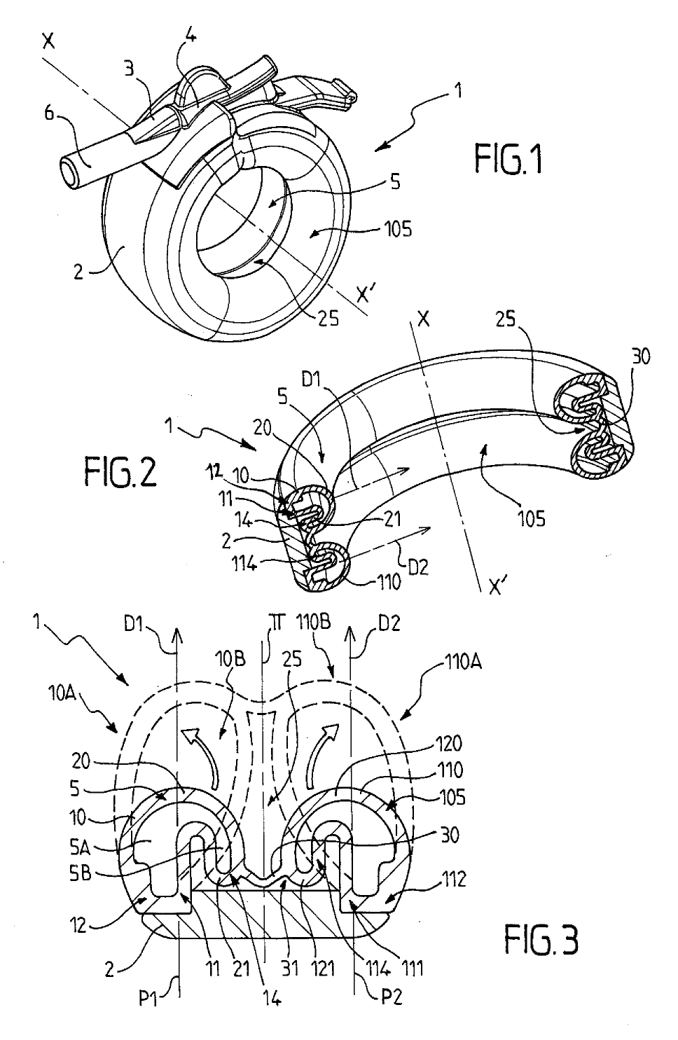

More particularly, the flexible band 2 and more generally the ring 1 are designed to be wound geometrically about a mean axis of extension (X-X′) as is already shown in FIGS. 1 and 2, said axis of extension (X-X′) advantageously coinciding, after implantation, with the main direction of extension of the duct or pocket formed by the biological organ.

Advantageously, the flexible band 2 is flexible enough to pass from an open configuration (not shown) to a closed configuration shown in FIG. 1, in which the junction means 3, 4 cooperate, for example by clip-on means, to close said flexible band 2 on itself appreciably at the level of its ends.

In this respect, said junction means 3, 4 may comprise a snug 3 projecting on to a rod linked to the first end and resting on the shoulder of a sleeve 4 fixed to the second end of said flexible band 2.

Preferably, the flexible band 2, or “bearing structure” is appreciably non-extensible in its main direction of extension, in such a way that once closed, it forms a bearing structure whose perimeter has an appreciably invariant length.

The ring according to the invention preferably constitutes a gastroplasty ring for the treatment of obesity and designed to be implanted around the stomach in order to reduce the diameter of the aperture of the stoma, or again to be implanted around the esophagus.

Naturally, the invention is in no way restricted to this application and pertains especially to surgical rings forming artificial sphincters adapted to the treatment of urinary or fecal incontinence, or again rings used to regulate the blood flow.

Depending on its intended use, the surgical ring 1 will naturally be adapted to the dimensions, surroundings and sensitivity of the biological organ concerned by the constriction, such as the bladder, the urethra, the intestine, the artery, the vein etc.

Advantageously, the surgical ring 1 has at least one first contact piece 5 that is designed to push against the biological organ and is capable of stretching into the ring 1 according to a appreciably radial first direction D1.

Thus, said first contact piece 5 advantageously projects into the interior of the closed loop formed by the ring in closed configuration and can undergo a appreciably radial, centripetal expansion towards the axis of extension (X-X′), so that the practitioner can adjust and especially restrict the aperture of the ring 1 through which the biological organ passes.

Thus, said first contact piece 5 and more generally the ring 1 can pass from a “contracted” configuration in which the first contact piece 5 is away from the axis (X-X′) at an initial distance, to a “deployed” configuration in which the first contact piece 5 approaches the axis (X-X′) at a distance smaller than its initial distance, and vice versa.

In a particular preferable way, the first contact means 5 can have an appreciably ring-shaped structure of axis (X-X′) and extend appreciably throughout the length of the flexible band 2 so as to be able to exert an adjustable constriction on the biological organ.

Naturally, the nature, shape and dimensions of the first contact piece 5 are in no way restrictive.

In particular, it can be envisaged that said first contact piece is made on an element of solid material, or again a sealed cavity filled with any fluid.

However, in a particularly preferred way, said first contact piece 5 will be formed by a first inflatable pocket designed to be filled with a filler fluid.

For ease of description, said first contact piece will be likened to a first inflatable pocket here below.

Advantageously, said first inflatable pocket 5 communicates for example by means of a catheter-type conduit 6 with a distant fluid transfer means (not shown) such as a syringe or an implanted container in such a way that the quantity of filler fluid contained in said pocket can easily be adjusted by injection or removal.

Thus, the first contact piece 5 can pass from a contracted configuration in which it contains a minimum volume of fluid and is represented by solid lines in FIG. 3 to a deployed configuration in which it contains a greater volume of fluid and is represented by dashes in this same FIG. 3.

Furthermore, said first contact means 5 may advantageously occupy any intermediate configuration between the two configurations, namely the contracted and deployed configurations, in such a way that they can carry out a appreciably continuous adjusting of the constriction of the biological organ in a predetermined range.

According to a major characteristic of the invention, the first contact piece 5 is demarcated by a first membrane 10, said first membrane 10 being provided with a deformable structure which is asymmetrical relative to the first direction D1 along which said first contact piece 5 may extend.

The term “provided with an asymmetrical deformable structure” means that the first membrane 10, especially when it is at rest, in a contracted configuration, has a constitution different in its part 10A situated “to the left” of the first direction D1 in FIG. 3, from the one that it has in the part 10B situated “to the right” of this same first direction D1.

Thus, the behavior in deformation of said membrane, under the effect of inflation by the addition of filler liquid (or conversely deflation by draining of said io fluid) is not symmetrical on either side of the line of separation indicating said first direction D1.

The result of this consequently is an asymmetrical deformation of the first inflatable pocket 5 which may advantageously be used to re-arrange the constituent material of said first membrane 10 during its deployment, and thus considerably limit the probability of formation of folds liable to cause the pinching of the wall of the biological organ.

Thus, by deliberately creating a controlled asymmetrical deformation of the first contact piece 5, the ring is given a particularly non-traumatic behavior.

Naturally, the structural differences between the “left-hand” part 10A and the “right-hand” part 10B of the first membrane 10 are in no way restrictive.

Thus, according to one preferred embodiment represented by FIGS. 2 and 3, these differences can be geometrical, with the membrane 10 having a layout and a spatial distribution different in its left-hand part 10A and its right-hand part 10B.

According to another alternative embodiment (not shown), these differences may lie in the nature of the constituent materials of each of said left-hand and right-hand parts, and more particularly in the intrinsic rigidity of said materials so as to encourage the deformation in the right-hand part 10B by means of a flexible material while limiting it in the left-hand part 10A by means of a more rigid material or vice versa.

Preferably, the deformable structure of the first membrane 10 is designed so that, during the expansion of the first contact piece 5 in the first appreciably radial direction D1, it gives rise to the joint migration of at least one part of the first membrane 10 from one side to the other of a median plane P1 of said first contact piece 5 that is normal to the axis of extension (X-X′).

In other words, the centripetal radial shift of the projecting part of the first membrane 10 is advantageously accompanied by a certain lateral deviation of at least one portion of said first membrane 10, in such a way that there is a transfer of the constituent material of said first membrane 10 from one side to the other of the median plane P1 along a component that is transversal to the first direction D1 and appreciably borne by the direction of the axis of extension (X-X′), i.e. at least one part of said first membrane 10 which is initially situated in the right-hand part 10B of this membrane, when the first contact piece 5 is in a contracted configuration, moves comprehensively until it meets the left-hand part 10A situated on the other side of the median plane P1 when the first contact piece 5 passes from its contracted configuration to its deployed configuration under the effect of the inflation.

Advantageously, this re-ordering of the spatial distribution of the constituent material of the first membrane 10 gives a certain rolling effect for said first membrane 10 along the surface of the biological organ, thus considerably limiting risks of abrasion or pinching of the wall of this biological organ.

Preferably, the first contact piece 5 is fixed to the bearing structure 2 respectively at the level of a first lateral junction zone 11 and a second lateral junction zone 12 which are distant from one another, the first membrane 10 being then folded down on itself on the first lateral junction 11 side when the first contact piece 5 is in a contracted configuration, so as to form a reserve of material 14 situated appreciably opposite the second lateral junction 12.

In other words, as illustrated in FIGS. 2 and 3, the first membrane 10 advantageously has an excess of material situated in its right-hand part 10B so that a part of said material can be transferred from one edge to the other, in this case from right to left, of the median plane P1 during the deployment of the first contact piece 5.

Preferably, the first and second junction zones form appreciably circular and parallel lines, with the axis (X-X′), by which the basis of the first inflatable pocket 5 is continuously fixed to the bearing structure 2.

Advantageously, the first and second lateral junction zones 11, 12 form appreciably invariant fixed points, relative to which the re-ordering of the membrane 10 is carried out during the inflation or emptying of the first inflatable pocket 5.

It must be noted that the reserve of material 14 according to the invention permits the deployment of the first contact piece 5 appreciably without creating any major tension at the first membrane 10, thus avoiding any distension or accidental cracks in this membrane.

Thus, a particularly robust structure is obtained that is not subjected to fatigue, so that the ring 1 has great longevity.

Preferably, as shown in FIG. 3, the first membrane 10, when the first contact piece 5 is in a contracted configuration, includes a first section 20 called a “smooth” section connected to the second lateral junction zone 12 and a second section 21 called a “folded”section connected to the first lateral junction zone 11, the first smooth section 20 meeting the second folded section 21 and forming a dome which covers at the same time said second folded section 21 and the first lateral junction zone 11.

Advantageously, this second folded section 21 is the above-mentioned reserve of material.

Furthermore, according to a preferred characteristic which may constitute an invention on its own, the membrane 10 thus advantageously has an asymmetrical structure capable of unwinding or on the contrary of getting wound on itself, especially in a lateral straightening motion or sagging motion respectively, borne by the axis (X-X′), during the centripetal radial deployment and the centrifugal radial withdrawal, respectively, of the contact piece 5.

Preferably, the first smooth section 20 forms a regular dome with a smooth surface that is convex relatively to the interior of the ring 1, a hemi-cylindrical surface for example, which separates the biological organ from the folded zone of the second section 21 in such a way that this zone 21 is not liable to come into direct contact with the wall of the biological organ when the ring is in the contracted configuration. Thus, the pinching of the biological organ at the second section 21 is advantageously avoided.

Furthermore, the first section 20 and the second section 21 preferably joined so as to form only one first single-piece membrane 10.

Naturally, the shape, layout and number of folds of the second section 21 is in no way restrictive.

However, according to one preferred embodiment which may constitute an invention as such, the layers formed by the folds of the second section 21 are appreciably stacked along planes normal to the mean axis of extension (X-X′), i.e. extending appreciably in parallel to the median plane P1 of the first contact piece 5.

Advantageously, this layout enables the storing of a reserve of material 14 in a space demarcated beneath the first smooth section 20, between said first section 20 and the bearing structure 2 forming the dorsal element of the ring.

Thus, the ring 1 at rest and especially when it is being implanted has a particularly compact structure.

In a particularly preferable way, the second folded section 21 has two folds which are laid out in an “S” reclining against the flexible band 2, in such a way that the first contact piece 5 is appreciably divided, when at rest in the contracted configuration, into a main filling cavity 5A and a reserve sub-cavity 5B, the latter having a volume that is initially smaller than the main cavity 5A and freely communicating with this cavity 5A.

It must be noted that, according to the preferred alternative embodiment illustrated in FIGS. 2 and 3, the first contact piece 5 has a particularly simple, light and compact structure which may in particular be easily obtained by molding.

Naturally, the first membrane 10 is made out of a material that is flexible enough to permit it to pass from the contracted configuration to the deployed configuration, for example a biocompatible elastomer material of the silicone type.

Preferably, the ring 1 also has a second contact piece 105 that is designed to push against the biological organ and is capable of extending into the interior of the ring 1 in a second appreciably radial direction D2, said second contact piece 105 being demarcated by a second membrane 110 provided with a deformable structure which is asymmetrical relative to the second direction D2.

Advantageously, the characteristics of the second contact piece 105, its shape, arrangement and its dynamic operation may be deduced, all things being equal, from the characteristics of the first contact piece 5.

For convenience of description, the references associated with the elements corresponding to this second contact piece 105 will correspond to the references associated with similar elements of the first contact piece 5 incremented by a value 100.

Thus, in particular, the second contact piece 5 is preferably formed by a second inflatable pocket demarcated by a second membrane 110 which is divided into a first smooth section 120 connected to a second folded section 121 which has a reserve of material 114 on one side of the median plane P2 of said second contact piece 105, when the latter is in the contracted configuration.

In a particularly preferred manner, as illustrated in FIGS. 2 and 3, and according to a characteristic which can constitute an invention on its own, the first contact piece 5 and the second contact piece 105 are mounted in opposition to each other on the bearing structure 2, in such a way that the first direction D1 and the second direction D2 are appreciably parallel and said first and second contact pieces 5, 105 can extend jointly while their respective deformable structures act in a appreciably antagonistic way.

More particularly, the first and second contact pieces 5, 105 are advantageously arranged in such a way that they can follow appreciably parallel radial expansions having the same sense, whereas in contrast, the tendency to lateral deviation of the first membrane 10 is exerted in a transversal sense that is appreciably opposite that of the deviation of the second membrane 110.

Thus, while the first membrane 10 tends to tilt from right to left during a deployment, as illustrated in the sectional view of FIG. 3, the second membrane 110 gets deployed on the contrary from left to right.

More particularly, the first and second inflatable pockets 5, 105 preferably have their respective reserves of material 14, 114 face to face in such a way that their contributions are appreciably opposite to each other.

Furthermore, in a particularly preferred way, the first and second contact pieces 5, 105 are both formed by ring-shaped pockets which are tiered in the extension axis (X-X′) and extend appreciably in parallel to each other.

Thus, advantageously, the ring 1 compliant with the invention presents, with the biological organ, a plurality of contact zones that are appreciably ring-shaped and disjoined from one another and more particularly actually superimposed at a distance from one another, giving the ring excellent stability.

More particularly, the first and second contact pieces 5, 105 may be separated by a depression 25 opening into the interior of the ring 1 and extending preferably throughout the length of the flexible band 2 in such a way that each of the contact pieces 5, 105 gives an independent circlip type anchoring around the biological organ.

Advantageously, each anchoring point is thus liable to hold the ring in position even when there is a temporary loss of adhesion of the other anchoring point, for example under the effect of peristaltic contractions of the stomach.

Furthermore, this physical separation of the contact zones also gives a greater range while at the same time reducing the effective surface of said contact zones between the ring and the biological organ, thus restricting risks of injury to the stomach wall, as well as the discomfort caused to the patient by the ring 1.

In a particularly preferred way, the first contact piece 5 and the second contact piece 105 are images of each other by plane symmetry, in this case with respect to the sagittal plane π of the ring 1 which cuts the flexible band 2 forming the bearing structure in its middle.

Advantageously, said sagittal plane π extends in parallel to the respective median planes P1, P2 of said first and second contact pieces 5, 105 and appreciably at mid-distance from them.

In a particularly advantageous way, the arrangement in opposition of the first and second contact pieces 5, 105, especially when the latter has structures that are images of each other, enables the overall symmetry of the ring 1 to be established along with its operation relative to the sagittal plane.

In other words, even if the individual structure of a contact piece is locally symmetrical, the combining of these two contact pieces restores the symmetry of the whole, thus preventing any imbalance during the adjusting and holding of the ring.

Furthermore, it is remarkable that, as understood in the invention, the first and second contact pieces 5, 105 can be formed respectively by a first and second inflatable pocket which communicate with each other and form the two projecting portions of a same chamber.

According to an alternative embodiment of this kind, the chamber forms a common container for the filler fluid and has two distinct projecting portions which constitute said gripping pockets 5, 105 and more particularly show a first and second peak portion projecting into the loop formed by the ring and designed to come into contact with the biological organ respectively at the level of a first contact zone and a second contact zone.

Advantageously, the first and second inflation pockets 5, 105 can be supplied by a same conduit 6 which connects them at the level of a common collector.

According to a preferred alternative embodiment, the first and second contact pieces 5, 105 are connected to each other by a bridge 30 distinct from the bearing structure 2.

Thus, said bridge 30 forms a kinematic link which joins the first inflatable pocket 5 to the second inflatable pocket 105 in straddling a material-free zone forming a cavity 31 which separates the first inflatable pocket from the second inflatable pocket.

Advantageously, the space left vacant by the cavity 31 and demarcated between the first and second inflatable pockets 5, 105 by the bridge 30 and the flexible band 2 forms a storage zone in which the first and second membranes 10, 110 can get folded when the ring is in the contracted configuration.

Preferably, the cavity 31 is in free communication with the exterior of the ring 1, by means of one or more unspecified holes, for example open at the ends of the flexible band 2. Thus, said cavity 31 is preferably laid out so that it cannot contain or retain a fluid under pressure, and especially a liquid and is capable of adapting spontaneously and passively to deformations of the membranes 10, 110.

Furthermore, the bridge 30 advantageously forms a kinematic holding means that limits the travel of the first inflatable pocket 5 relatively to the second inflatable pocket 105 and more specifically the antagonistic lateral clearance of the first and second membranes 10, 110, during the joint radial expansion of these two membranes.

Thus, the bridge 30 advantageously balances the lateral expansion of said membranes in keeping the first and second contact pieces 5, 105 appreciably parallel to each other, these contact pieces holding each other mutually in such a way that the antagonistic side motions of their respective membranes 10, 110 appreciably compensate for each other.

In other words, the bridge 30 prevents excessively out-projecting lateral movements of either of the inflatable pockets on either side of the ring, in limiting the tilting of their respective smooth sections 20, 120 which provides for even and combined development of the set formed by the first and second contact pieces 5, 105.

Preferably, the attachment fixtures of the apron of the bridge 30 correspond to the joining zones between the first smooth section 20, 120 and the second folded section 21, 121 of each of the first and second membranes 10, 110.

Furthermore, the bridge 30 can advantageously show an inflection zone, for example a V-shaped zone, the tip of which is pointed to the flexible band 2 as illustrated in FIG. 3, so that it can get elastically deformed like a hinge and thus adapt to variations in distance between the first membrane 10 and the second membrane 110 during the radial deployment or contraction of these membranes.

The working of a ring compliant with the one shown in FIGS. 1 to 3 shall now be described.

First of all, the practitioner introduces the open ring, for example by laparoscopy, and engages it around the biological organ.

He then incurvates the bearing structure 2 until it is closed on itself to form a loop gripping the biological organ and locks the bearing structure 2 at its ends by engaging the snug 3 into the sleeve 4.

When he does so, the peak portions of the first and second contact pieces 5, 105 get applied in a resting position against the wall of the biological organ, respectively on a first and second appreciably ring-shaped contact zone situated at a distance along the axis of extension (X-X′).

At rest, i.e. before the filling of the first and second inflatable pockets, forming the first and second contact pieces 5, 105, said pockets are in a contracted configuration as shown in solid lines in FIG. 3.

More particularly, it is the projecting domes formed by the respective first smooth sections 20, 120 of the first and second membranes 10, 110 that then present a appreciably semi-cylindrical domed profile for which the peak lines come into contact with the biological organ.

Preferably, in a section of the ring, the culminating point belonging to the peak line of each first smooth section 20, 120, i.e. the point of the external surface of the membrane 10, 110 that is closest to the extension axis (X-X′), is situated so as to be appreciably vertical to the first lateral junction zone 11, 111 of the corresponding contact piece.

It is noteworthy that the volume of the cavity 31 is then particularly reduced, with the bridge 30 being in the immediate vicinity, or even in partial contact with the bearing structure 2.

When the practitioner inflates the first and second inflatable pockets 5, 105 i.e. when he introduces an appropriate filler fluid into these pockets, the increase in pressure and/or volume resulting from this injection forces the centripetal radial deployment of said inflatable pockets.

This inflation takes the form of a gradual and appreciably simultaneous deployment of the first and second membranes 10, 110 in such a way that their peak portion gradually moves away from the bearing structure 2 along a first and second appreciably radial, centripetal and mutually parallel direction D1, D2.

This overall centripetal radial progression is accompanied by a tilting of the first smooth sections 20, 120 which sag towards the free lateral edges of the ring 1 as illustrated by the arrows in FIG. 3, in such a way that said first smooth sections 20, 120 are gradually straightened, while their radius of curvature increases and they move away gradually from the sagittal plane π of the ring.

Thus, according to one characteristic which may constitute an invention in its own right, each deformable structure according to the invention is preferably laid out so as to prompt the tilting of the respective membrane 10, 110 during the centripetal radial deployment of this membrane.

More particularly, the first and second contact piece 5, 105 are therefore preferably laid out in such a way that their centripetal radial deployment is accompanied by an unrolling of their respective membranes 10, 110 towards the lateral edge of the ring which is the closest to them.

Advantageously, this respective rolling out of each of the pockets to the lateral edge of the ring that is the closest to it tends to limit the relative shift of the membrane 10, 110 in relation to the wall of the biological organ, and prompts especially a relative shift by a rolling rather than by friction in such a way that the deployment is particularly careful with the tissues of said biological organ.

Referring to FIG. 3, the first membrane 10 tends to tilt on the whole from right to left, its first section 20 shifting laterally in gradually crossing the median plane P1 while the second membrane 110 tends to follow a appreciably opposite path from left to right.

Advantageously, the material stretch created by this lateral opening is simultaneously compensated for by the gradual unfolding of the second folded sections 21, 121 which recline in gradually opening the constituent folds of their reserves of material 14, 114.

The deployment can advantageously continue until the reserves of material 14, 114 are absorbed, i.e. until the initially folded second sections 21, 121 are straightened.

In this respect, it is noteworthy that the reserves of material 14, 114 advantageously make it possible to deploy the inflatable pockets 5, 105 without having to distend and therefore fragilize their respective membranes 10, 110. This increases the resistance and longevity of the ring.

Advantageously, the bridge 30 keeps a transversal kinematic link between first and second membranes 10, 110 and therefore puts up a mutual hold-back stress against the free axial play, and more particularly to the moving apart, in a appreciably corolla-like opening motion, of the first and second inflatable pockets 5, 105 along the axis of extension (X-X′).

In other words, the bridge 30 advantageously permits the antagonistic unrolling of the first and second membranes 10, 110 while maintaining the first and second contact piece 5, 105 in a appreciably grouped and parallel position during their radial deployment, thus providing for an even deployment of the ring 1 until it reaches its configuration of functional constriction and for example its configuration of maximum deployment as illustrated in dashes in FIG. 3.

Besides, it is noteworthy that during the deployment of the first and second membranes 10, 110, the bridge 30 gets separated from the bearing structure 2 and is driven in a centripetal radial shift by the deformation of said first and second membranes 10, 110 without putting up significant resistance to this motion.

Furthermore, the bridge 30, especially because of its flexible inflection zone, advantageously permits axial play to the precise level necessary and sufficient for the efficient deployment of the inflatable pockets and may especially get adapted at any time to the residual width of the depression 25 which remains between said pockets.

Furthermore, said bridge 30 advantageously remains withdrawn from the peak portions of the contact pieces 5, 105 especially when these peak portions are deployed to the maximum, i.e. as close as possible to the axis of extension (X-X′), in such a way that the dual anchoring of the ring 1 is always operational and keeps this ring in a high state of stability.

Naturally, all the description elements provided in the context of the centripetal radial deployment of the contact pieces 5, 105 remain applicable, all things being equal, to the centrifugal radial contraction of said contact pieces 5, 105.

Thus, when the practitioner deflates the inflatable pockets 5, 105 so as to make them return from their deployed configuration to their contracted configuration, i.e. so as to increase the diameter of constriction of the biological organ, the peak portions of said pockets collapse in “redescending” toward the bearing structure 2 in a centrifugal radial motion and draw the bridge 30 along with them, which has the effect of gradually crushing the cavity 31.

More particularly, the respective first sections 20, 120 of the inflation pockets 5, 105 sag and tilt in order to come closer to the sagittal plane π, in re-crossing the median planes P1, P2 of the first and second inflatable portions 5, 105 in reverse, in a winding movement while the second sections gradually absorb the surplus material in getting folded.

In this respect, the intrinsic elasticity of the second sections 21, 121 advantageously gives a shape memory to the first and second membranes 10, 110.

Secondly, during this collapse, the bridge 30 acts as a return spring which tends to bring closer together the first and second membranes 10, 110 situated so as to be facing each other on either side of the depression 25.

Advantageously, these effects can get combined to facilitate the ordered and gradual folding of said second sections 21, 121 until the reconstitution of the reserves of material 14, 114 in the contracted configuration, the first and second membranes 10, 110 getting gradually folded so as to be “stacked” beneath their respective first sections 20, 120, thus making it possible to “store” said reserves of material 14, 114 appreciably at the centre of the flexible band.

It is noteworthy that again neither the bridge 30 nor the cavity 31 put forward any significant counter resistance to this centrifugal radial motion so that the first and second gripping pockets 5, 105 can return, especially by elastic return, to any intermediate position up to their initial contracted position.

Thus, the ring 1 compliant with the invention has a dynamic behavior that is particularly respectful of the biological organ since it limits friction against the wall of said biological organ while at the same time preventing the pinching of said wall both during the deployment and the contraction of the contact pieces 5, 105.

Furthermore, the passage from the contracted configuration to the deployed configuration or vice versa is advantageously regulated and reproducible, with the membranes following appreciably predictable, controlled and reversible paths.

Besides, the ring 1 of the invention has an open-worked structure that is particularly light and robust which, in addition, undergoes little fatigue stress, in such a way that that the ring 1 has great longevity.

Finally it is noteworthy that the ring 1 of to the invention enables a precise, continuous and fine adjusting of the constriction while at the same time permanently preserving its qualities of comfort and dynamic stability.

### POSSIBILITY OF INDUSTRIAL APPLICATION

The invention can be applied on an industrial scale to the designing and manufacture of surgical rings, especially gastric rings.

1. Implantable surgical ring configured to be placed around a biological organ constituting a pocket or a duct, in getting wound around a mean axis of extension in order to modify the section of passage of said biological organ, said ring comprising at least one first contact piece configured to push against said biological organ and being capable of extending into the interior of the ring along a first appreciably radial direction, said first contact piece being demarcated by a first membrane provided with a deformable structure asymmetrical relative to said first direction and configured, during the expansion of the first contact piece along the first appreciably radial direction, to bring about the joint migration of at least one part of the first membrane from one side to the other in a median plane of said first contact piece perpendicular to the axis of extension.

2. Surgical ring according to claim 1, wherein the deformable structure is configured to prompt the lateral tilting of the membrane during the centripetal radial deployment of this membrane.

3. Surgical ring according to claim 1, further comprising a bearing structure to which the first contact piece is fixed respectively at the level of a first lateral junction zone and a second lateral junction zone which are distant from one another, wherein the first membrane is folded down on itself on the first lateral junction side when the first contact piece is in a contracted configuration, so as to form a reserve of material situated appreciably opposite the second lateral junction.

4. Surgical ring according to claim 3, wherein the first membrane, when the first contact piece is in contracted configuration, comprises a first “smooth” section connected to the second lateral junction zone and a second “folded” section connected to the first lateral junction zone, the first smooth section meeting the second folded section and forming a dome which covers at the same time said second folded section and the first lateral junction zone.

5. Surgical ring according to claim 1, further comprising a second contact piece that is configured to push against the biological organ and is capable of extending into the interior of the ring in a second appreciably radial direction, said second contact piece being demarcated by a second membrane provided with a deformable structure which is asymmetrical relative to the second direction.

6. Surgical ring according to claim 5, further comprising a bearing structure on which the first and second contact pieces are mounted in opposition to each other in such a way that the first direction and the second direction are appreciably parallel and that said first and second contact pieces can extend jointly while their respective deformable structures act in a appreciably antagonistic way

7. Surgical ring according to claim 6, wherein the first and second contact pieces are connected to each other by a bridge distinct from the bearing structure.

8. Surgical ring according to claim 5, wherein the first and second contact pieces are both formed by ring-shaped pockets that are tiered in the extension axis and extend appreciably in parallel to each other.

9. Surgical ring according to claim 8, wherein the first and second contact pieces are configured in such a way that their centripetal radial deployment is accompanied by an unrolling of their respective membranes towards the respective lateral edge of the ring which is closest to each of them.

10. Surgical ring according to claim 5, wherein the first contact piece and the second contact piece are images of each other by plane symmetry.

11. Surgical ring according to claim 1, wherein the surgical ring constitutes a gastroplasty ring designed for the treatment of obesity.

12. Surgical ring according to claim 1, wherein the first contact piece has an appreciably ring-shaped structure with the axis.

13. Surgical ring according to claim 3, wherein the first contact piece extends appreciably throughout the length of the bearing structure.

14. Surgical ring according to claim 13, wherein the first contact piece comprises a first section configured to come into contact with said biological organ, said first section forming a regular dome with a surface that is smooth and convex relatively to the interior of the ring.
